(19) United States
(12) Patent Application Publication
(10) Pub. No.: US 2011/0079227 A1
(43) Pub. Date: Apr. 7, 2011

(54) Pressure regulation mechanism for inflatable in-ear device

(76) Inventors: Michael C. Turcot, Montreal (CA); Jérémie Voix, Montreal (CA)

(21) Appl. No.: 12/923,696

(22) Filed: Oct. 5, 2010

Related U.S. Application Data
(60) Provisional application No. 61/272,534, filed on Oct. 5, 2009.

(51) Int. Cl. A61F 11/10; H04R 25/00

(52) U.S. Cl. 128/865; 381/328

(57) ABSTRACT

An inflatable in-ear device incorporates a pressure regulating mechanism, for example a simple length of tubing or a relief/unloader valve, at the inflow or outflow from the inflating sheath inserted within the ear canal, thus allowing pressure regulation of the settable compound being injected for inflation of the sheath while avoiding excesses in pressure in the inflating compound and ensuring the comfort of the individual.

### CROSS REFERENCE TO RELATED APPLICATIONS

Benefit of U.S. Provisional Application for Patent Serial No. 61/272,534 filed on Oct. 5, 2009, being incorporated herein by reference, is hereby claimed.

### FIELD OF THE INVENTION

The present invention concerns an inflatable in-ear device and in particular has reference to a component thereof for modulating and controlling the inflation pressure during inflation.

In-ear devices include intra-aural hearing protectors (earplugs), earphones, hearing-aid devices and the like and the invention relates to custom-fitting in-ear devices that are formed in situ to conform morphologically to the inside of the ear canal and the cavum concha of the individual.

### BACKGROUND OF THE INVENTION

The present inventors have proposed hearing protection devices which offer custom-fitting to the ear of the individual and are exemplified and claimed in U.S. Pat. Nos. 6,339,648 and 6,754,357 to McIntosh at al. Principally, these devices comprise a core member around which is arranged a sheath in a deflated state, a permanent setting compound being injected into the cavity defined between the core member and the sheath to bring the device into morphological conformity to the contours of the ear canal. In this way, the in-ear device is produced in customized fashion thus enhancing the performance thereof and indeed the comfort of the individual.

The sheath is typically produced from a silicon material having hydrodynamic properties enabling a quasi-isobaric inflation process which maintains a substantially constant pressure (P1) during inflation without external constraints (similarly to an inflatable balloon), which could typically be in the order of about 0.8 psig (about 5,5 kPa relative pressure). However, when constrained within the ear canal, the internal pressure rises and causes the in-ear device to overinflate and may injure or cause discomfort to the wearer of the device (at a pressure above a comfort-limit of ear canal pressure level P2) when a settable compound is injected between the core and the sheath through an injection channel, which is in the order of about 2.0 psig (about 14 kPa relative pressure). U.S. Pat. No. 6,687,377 and in U.S. patent application Ser. No. 2005-0123146A1 tentatively try to solve that problem of limiting the maximum pressure level by assessing in situ the acoustic attenuation of the in-ear device during the inflation mode.

Accordingly, there is a need for an improved mechanism capable of regulating the pressure during the inflation mode of an inflatable in-ear device.

### SUMMARY OF THE INVENTION

A general object of the present invention is thus to provide a mechanism capable of regulating the pressure during the inflation mode of an inflatable in-ear device.

An advantage of the present invention is that the pressure regulating mechanism has a maximum pressure limit set at a value that ensures the in-ear device morphologically conforms to the inside of the ear canal and the cavum concha of the individual without discomfort.

Another advantage of the present invention is that the pressure regulating mechanism ensures that the quantity of compound delivered for the adaptation of the in-ear device to the geometry of a human ear canal is sufficient to allow proper expansion to assume the contour of the ear canal and the cavum concha of the individual.

A further advantage of the present invention is that a facility is provided for limiting the amount of settable compound delivered for inflation of the sheath for the purpose of conforming morphologically to the ear canal.

Yet another advantage of the present invention is that the pressure regulating mechanism includes a setting pressure mechanism that enables the user to vary the maximum pressure limit to ensure a better noise protection from the inflatable in-ear device wherever required, even though some discomfort could be tolerated by the user for short period of times.

According to an aspect of the present invention, there is provided a pressure regulating mechanism for regulating the pressure of a settable compound being injected into an inflatable in-ear device, said in-ear device including a core member of generally similar form to the contour of the ear canal of an individual, a sound bore being defined within and through the core member from a region exterior to the ear canal to a relatively inner region within the ear canal, a platform to which the core member is attached exteriorly of the ear canal, an inflatable open-ended sheath having a closed end secured to the relatively inner region of the core member, the sheath assuming a first condition and being foldable into an inflatable condition in close adjacency to the core member prior to inflation, the core member and the platform having communicating injection channels for injecting a settable compound into a space defined between the core member and the sheath in its inflatable condition, said pressure regulating mechanism comprising a main body connecting to an outflow channel communicating with the space, said main body allowing discharge of any excess settable compound while maintaining the pressure of the settable compound at a predetermined pressure level below a comfort-limit pressure level acceptable inside the ear canal of the individual during inflation of the sheath from the inflatable condition up to and at an inflated condition with the sheath substantially assuming an inside contour of the ear canal.

According to another aspect of the present invention, there is provided an inflatable in-ear device comprising a core member of generally similar form to the contour of the ear canal of an individual, a sound bore being defined within and through the core member from a region exterior to the ear canal to a relatively inner region within the ear canal, a platform to which the core member is attached exteriorly of the ear canal, an inflatable open-ended sheath having a closed end secured to the relatively inner region of the core member, the sheath assuming a first condition and being foldable into an inflatable condition in close adjacency to the core member prior to inflation, the core member and the platform having communicating injection channels for injecting a settable compound into a space defined between the core member and the sheath in its inflatable condition, an outflow channel communicating with the space allowing discharge of any excess settable compound, and a pressure regulating mechanism associated with the outflow channel.

Conveniently, the pressure regulating mechanism may function also as a mechanism for limiting the amount of settable compound injected into the said space.

The main body of the pressure regulating mechanism may be in the form of tubing of sufficient length as to secure pressure regulation of the compound within the void and to allow discharge of any excess compound issuing forth from the outflow channel.

In the alternative, the main body of the pressure regulating mechanism may be in the form of tubing provided with an external pressure applicator enabling constriction thereof to modulate the pressure and the flow therethrough. For example, the external pressure applicator may be in the form of a screw clamp which upon actuation effects a change in the bore size of the tubing thereby effecting a variation of flow therethrough. It will be appreciated by the skilled addressee that the external regulator may be of any suitable form to achieve the same result.

In a further alternative, the main body of the pressure regulating mechanism may be in the form of a relief or unloading valve enabling the modulation of pressure and control of the amount of material within the void between the sheath and the core member during inflation and prior to setting.

Other objects and advantages of the present invention will become apparent from a careful reading of the following description.

### BRIEF DESCRIPTION OF THE DRAWINGS

Further aspects and advantages of the present invention will become better understood with reference to the description in association with the following figures, in which similar references used in different figures denote similar components, wherein:

FIG. 1 is a partial schematic cross-sectional view of an inflatable in-ear device with a first embodiment of pressure regulating mechanism in accordance with the present invention;

FIG. 2 is a view similar to FIG. 1 showing a second embodiment of pressure regulating mechanism in accordance with the present invention;

FIG. 3 is a view similar to FIG. 1 showing a third embodiment of pressure regulating mechanism in accordance with the present invention; and

FIG. 4 is a view similar to FIG. 1 showing a fourth embodiment of pressure regulating mechanism in accordance with the present invention.

### DETAILED DESCRIPTION OF THE PREFERRED EMBODIMENTS

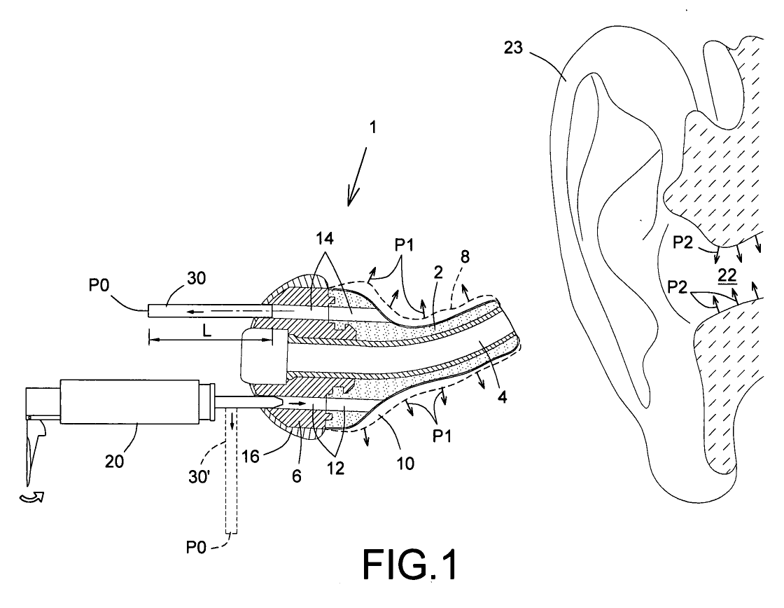

Referring to the drawings there is illustrated in FIGS. 1, 2, 3 and 4 an inflatable in-ear device 1 comprising a core member 2 with a sound bore 4 extending therethrough and into a platform 6 to which the member 2 is attached. A sheath 8 of flexible material surrounds the core member 2 (shown non-uniformly spaced from the core member 2, as if separated therefrom by a compound material, for the purpose of clarity) and provides a space 10 for the injection of a settable compound (not shown but inside body of 20), such as a silicone compound or the like. The core member 2 and the platform 6 are both provided with injection and outflow channels 12, 14 for respectively allowing the ingress and the egress of the compound into and out of the space 10. The platform 6 is provided with an end capping 16 which is so formed as to allow access to the inflow and outflow channels. An injection syringe 20 is shown in the figures as the means of injecting the compound in use into the space 10, but it will be understood that other means may be employed. The device 1 is shown poised for insertion into the ear canal 22 of an individual whose ear 23 is illustrated.

Generally, the inflatable in-ear device 1 is duly inserted into the ear canal 22 and the settable compound (not shown) is injected by use of the syringe 20 through the injection channel 12 to inflate the sheath 8 and fill the space 10, between the sheath 8 and the core member 2, thereby causing the sheath 8 to conform morphologically to the contour of the ear canal 22. When the pressure of the compound reaches the appropriate predetermined pressure level P0, the compound will discharge through the outflow channel 14 via a pressure regulator mechanism.

In the first embodiment of FIG. 1 the pressure regulation mechanism has a main body in the form of a length ‘L ’of tubing 30 which is typically sufficient to receive to whole amount of settable compound, and therefore any possible excess thereof. The predetermined pressure PO is maintained substantially constant during the flow of the settable compound inside the tubing 30, since the pressure PO is the pressure required to displace the compound therealong in a so-called, in fluid dynamics, creeping flow, and essentially depends on physical characteristics such as the viscosity of the compound, the nature of the material and the internal diameter of the tubing. The predetermined pressure level PO is obviously larger than the pressure level P1 required to inflate the sheath 8 (level P1 depends essentially on the material of the sheath and its thickness) and smaller than the pressure level P2 at which an individual may start feeling discomfort into the ear canal 22, as mentioned hereinabove, to allow the inflation of the sheath while preventing overinflation thereof.

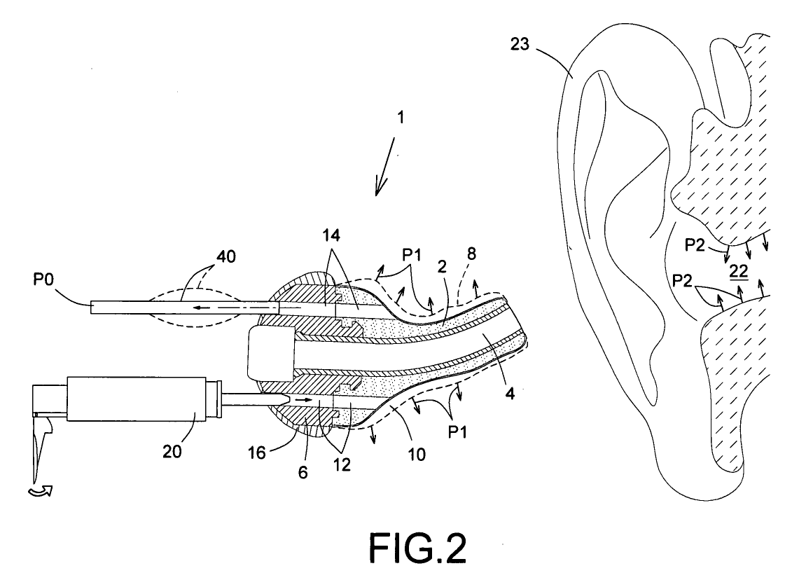

In the second embodiment shown in FIG. 2 the main body of the pressure regulating mechanism comprises an inflatable bladder 40 for receiving excess settable compound. The bladder 40, made out of any suitable material, is operable as a second external sheath having quasi-isobaric inflation pressure level P0 larger than P1 but lower than P2, the bladder inflating thereby to act as a valve to relieve pressure and to allow discharge of excess compound.

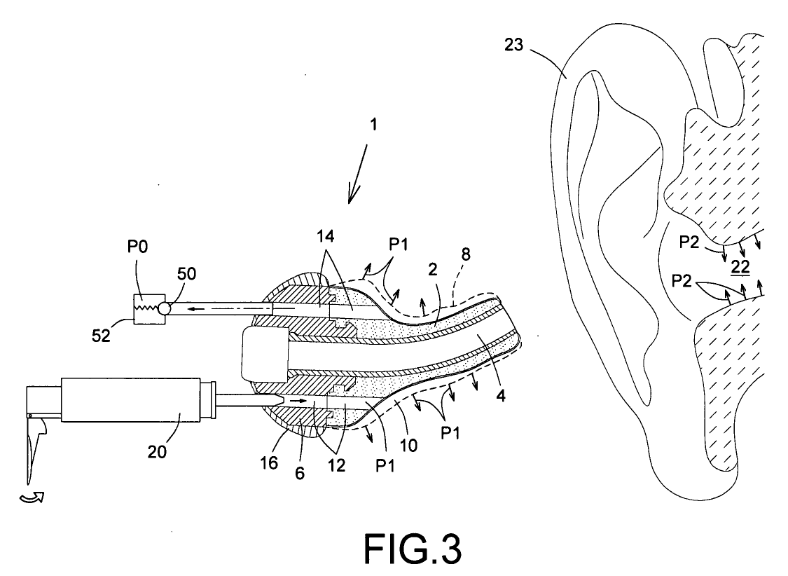

The third embodiment of the main body of the pressure regulating mechanism is shown in FIG. 3 and essentially comprises a relief/unloader valve 50 mounted on a reservoir 52. When the pressure reaches the predetermined level P0, the valve 50 will open and excess compound will be discharged into the reservoir 52, thus ensuring that the pressure in the sheath 8 does not exceed a level P2 acceptable to the individual and that the amount of compound is not excessive for the comfort of the individual.

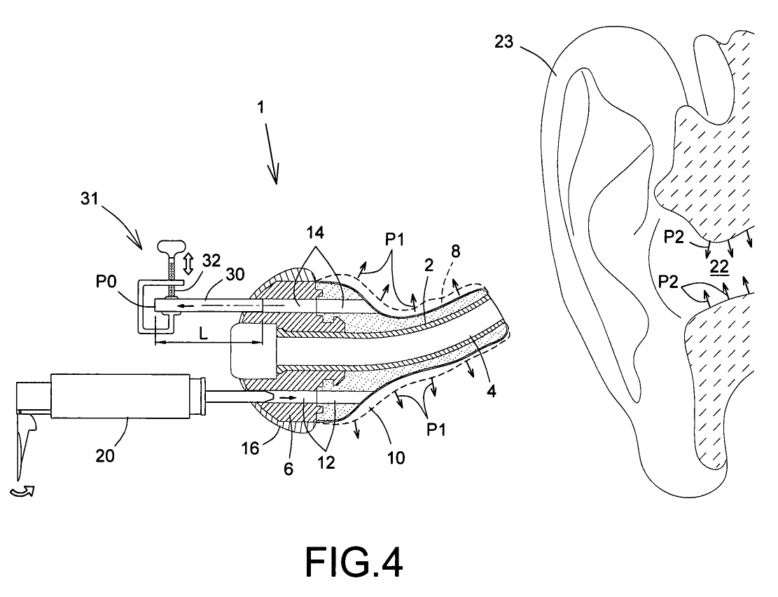

The fourth embodiment of the main body of the pressure regulating mechanism is shown in FIG. 4 and essentially comprises an external setting pressure applicator 31 which in this example is in the form of a screw clamp 32. The applicator 31 enables the user to vary the maximum pressure limit to ensure a better noise protection from the inflatable in-ear device wherever required by restricting the passage within the tubing 30 to modulate pressure and flow therethrough. For example, such variation is useful in applications such as in shooting training facilities for security forces, or in the military environment among others, even though some discomfort could be tolerated by the user for short periods of times.

Although the four embodiments of pressure regulating mechanisms in FIGS. 1 through 4 are shown located substantially downstream of the space 10 in communication with the outflow channels 14, it would be obvious to one skilled in the art that it could be located upstream of the space 10 (and downstream of the syringe 20), as illustrated in dotted lines in FIG. 1 for the length of tubing 30′ with an equivalent outflow channel, without affecting its function, within the scope of the present invention.

Although the present invention has been described with a certain degree of particularity, it is to be understood that the disclosure has been made by way of example only and that the present invention is not limited to the features of the embodiments described and illustrated herein, but includes all variations and modifications within the scope and spirit of the invention as hereinafter claimed.

We claim:

1. A pressure regulating mechanism for regulating a pressure of a settable compound being injected into an inflatable in-ear device, said in-ear device including a core member of generally similar form to the contour of the ear canal of an individual, a sound bore being defined within and through the core member from a region exterior to the ear canal to a relatively inner region within the ear canal, a platform to which the core member is attached exteriorly of the ear canal, an inflatable open-ended sheath having a closed end secured to the relatively inner region of the core member, the sheath assuming a first condition and being foldable into an inflatable condition in close adjacency to the core member prior to inflation, the core member and the platform having communicating injection channels for injecting a settable compound into a space defined between the core member and the sheath in its inflatable condition, said pressure regulating mechanism comprising a main body connecting to an outflow channel communicating with the space, said main body allowing discharge of any excess settable compound while maintaining the pressure of the settable compound at a predetermined pressure level below a comfort-limit pressure level acceptable inside the ear canal of the individual during inflation of the sheath from the inflatable condition up to and at an inflated condition with the sheath substantially assuming an inside contour of the ear canal.

2. A pressure regulating mechanism according to claim 1, wherein the main body of the mechanism is in the form of tubing of sufficient length as in use to secure pressure regulation of the compound within the void and to allow discharge of any excess compound issuing forth from the outflow channel.

3. A pressure regulating mechanism according to claim 1, wherein the main body of the mechanism is in the form of tubing, and an external pressure applicator is provided and is adapted to effect constriction of the tubing thereby to modulate pressure and flow therethrough.

4. A pressure regulating mechanism according to claim 3, wherein the external pressure applicator is in the form of a screw clamp adapted to apply restrict the passage within the tubing thereby to modulate pressure and flow therethrough.

5. A pressure regulating mechanism according to claim 1, wherein the body of the mechanism is in the form of an inflatable bladder.

6. A pressure regulating mechanism according to claim 1, wherein the main body of the mechanism is in the form of a relief valve adapted to modulate pressure and to control the amount of material within the void between the sheath and the core member during inflation and prior to setting.

7. A pressure regulating mechanism according to claim 1, wherein the main body of the mechanism is in the form of an unloader valve adapted to modulate pressure and to control the amount of material within the void between the sheath and the core member during inflation and prior to setting.

8. An inflatable in-ear device comprising a core member of generally similar form to the contour of the ear canal of an individual, a sound bore being defined within and through the core member from a region exterior to the ear canal to a relatively inner region within the ear canal, a platform to which the core member is attached exteriorly of the ear canal, an inflatable open-ended sheath having a closed end secured to the relatively inner region of the core member, the sheath assuming a first condition and being foldable into an inflatable condition in close adjacency to the core member prior to inflation, the core member and the platform having communicating injection channels for injecting a settable compound into a space defined between the core member and the sheath in its inflatable condition, an outflow channel communicating with the space allowing discharge of any excess settable compound, and a pressure regulating mechanism according to claim 1 associated with the outflow channel.

9. An inflatable in-ear device according to claim 8, wherein the pressure regulating mechanism is located downstream of the space defined between the core member and the sheath.

10. An inflatable in-ear device according to claim 8, wherein the pressure regulating mechanism is located upstream of the space defined between the core member and the sheath.
